(19) United States
(12) Patent Application Publication
(10) Pub. No.: US 2007/0255239 A1
(43) Pub. Date: Nov. 1, 2007

(54) Excrement disposal device

(75) Inventor: Sadao Hataya, Nerima-ku (JP)

Correspondence Address: JORDAN AND HAMBURG LLP, 122 EAST 42ND STREET, SUITE 4000, NEW YORK, NY, 10168, US

(73) Assignee: Musashi Co., Ltd., Nerima-ku (JP)

(21) Appl. No.: 11/796,897

(22) Filed: Apr. 30, 2007

(30) Foreign Application Priority Data
May 1, 2006 (JP) 2006-127506

(51) Int. Cl. A61M 1/00

(52) U.S. Cl. 604/319; 141/369

(57) ABSTRACT

An excrement disposal device includes a pair of base plates, a pair of wing plates and a disposal bag. Front and rear ends of the two base plates are pivotally attached to each other, to make an opening between the base plates variable. The wing plates are mounted on the two base plates. A top section of each wing plate is opposedly situated to face the aforementioned opening from ahead and from behind to longitudinally control the shape of the opening, thereby making the dimensions of a mouth section variable. The disposal bag is vertically inserted into the mouth section, wherein a mouth periphery of the inserted disposal bag touches a subanal area from below and can be opened and closed in accordance with the opening and closing motion of the anus.

### BACKGROUND OF THE INVENTION

1. Field of the Invention

The present invention relates to a handy-type excrement disposal device, and more particularly to an excrement disposal device which can be carried for use in outdoor activities, nursing-care services, military services and the like.

2. Description of the Prior Art

(Technological Background)

A diaper is a major example of an excrement disposal device of this kind. Various kinds of diapers have been developed recently for portable use and for nursing-care services.

However, the conventional excrement disposal devices of this kind have disadvantages in that an object (i.e., body waste; a major example is feces, hereinafter referred to as “object”) adheres to a body after use and it also gives out a bad odor. No excrement disposal device which has overcome these disadvantages has been found.

(Description of the Prior Art)

As a result of assiduous research and efforts, the present inventor has achieved an invention as described in the following Patent Document 1 and has filed an application for a patent. FIG. 8 is a perspective view of its application.

A conventional excrement disposal device 1 according to this Patent Document 1 is formed in a short, rectangular cylinder shape and has two (right and left) frames 2 and two (front and rear) frames 3. A sponge 4 is disposed within the frames. In this excrement disposal device 1, the front and rear ends of the right and left frames 2 and the right and left ends of the front and rear frames 3 are pivotally attached to each other on a vertical axis, while the front and rear frames 3 are also pivotally attached in the center on a vertical axis. The sponge 4 is vertically provided with a circular through-hole 5. Attached to the outer peripheral ends of the device 1, that is, to the right and left frames 2 and the front and rear frames 3, is a disposal bag 6.

In use, the outside of the right and left frames 2 is held by two fingertips A and the center (on the lower side of the figure) of the excrement disposal device 1 is caused to push against a subanal area. The front and rear frames 3 are then opened or closed in a substantially V-shape in accordance with the opening and closing motion of the anus to cause the excrement disposal device 1 to laterally expand or reduce as a whole, thereby expanding, reducing or closing the circular through-hole 5 of the sponge 4. The circular through-hole 5 is provided to form a mouth section 7.

In this manner, an object from the anus is housed in the disposal bag 6 through the mouth section 7 of a substantially circular ring shape adapted to match the shape of the anus. The conventional excrement disposal device 1 is used in this manner and is intended to dispose of the object without adhering to the body and without sending out a bad odor.

(Conventional Technical Literature Information)

[Patent Document 1] Japanese Unexamined Patent Publication No. 2003-175065

### DISCLOSURE OF THE INVENTION

3. Problems to be Solved by the Invention

It has been pointed out that the conventional excrement disposal device 1 has the following problems.

(First Problem)

An object sometimes escapes from the excrement disposal device 1 because it is not easy to correctly apply the center of the device 1 to a subanal area for positioning.

In use, it is necessary to correctly bring the center of the excrement disposal device 1, the center of the circular through-hole 5 of the sponge 4, that is, the center of the mouth section 7, in line with the center of the anus for positioning. However, in such a conventional excrement disposal device 1, this positioning operation is not easy and it is often the case that the operation is not conducted properly.

This is caused by the fact that the fingertips A required to play a major role in such an operation are outside the right and left frames 2 of the excrement disposal device 1, wherein the fingertips A do not reach the anus directly and are away from the point to be positioned. In other words, in the conventional excrement disposal device 1, it is not possible to conduct a confirming and positioning operation based on the direct feel or tactile impression and as a result, an indirect operation or a trial-and-error operation is often conducted. In addition, the skin of the anus periphery is soft and may have partly projected. These can also be considered as additional causes.

In this manner, in the conventional excrement disposal device 1 of this kind, it is not possible to grope for and pinpoint the location of the anus accurately. It is therefore often the case that the device 1 is pushed against the anus in the condition in which the center of the device 1 and the mouth section 7 deviate from the anus, and a gap is caused between the mouth section 7 and the anus.

In the conventional excrement disposal device of this kind 1, an accident whereby an object escapes from the circular through-hole 5, the mouth section 7, and the disposal bag 6 can occur. It has also been pointed out that problems occur whereby the object adheres to the human body or the fingertips and gives out a bad odor.

(Second Problem)

The conventional excrement disposal device of this kind 1 has a disadvantage in that the object often leaks out due to the unstable operation of the device.

Even though the center of the excrement disposal device 1 and the center of the mouth section 7 can be correctly positioned to face the center of the anus, it is often the case that the mouth section 7 does not always open or close surely in accordance with the opening and closing motion of the anus in the subsequent use and the object often leaks out. Even from this point of view, it is pointed out that the conventional excrement disposal device 1 has a disadvantage in that the object adheres to a human body and unpleasant odors are produced.

This results from the fact that the mouth section 7, that is, the circular through-hole 5 is formed by the sponge 4.

More specifically, since it is not possible to surely push the anus periphery with the soft sponge 4, it is often the case that the front and rear frames 3 and the excrement disposal device 1 do not move as expected in conjunction with the opening and closing motion of the anus. The sponge 4 can not surely follow the opening and closing motion of the anus and thus, the front and rear frames 3 and the excrement disposal device 1 do not open or close and expand or contract as expected through the sponge 4. In this manner, the mouth section 7 can not be opened or closed in a substantially circular shape to match the shape of the opening and closing anus and as a result, a displacement or a gap is often produced.

Even from this aspect, in the conventional excrement disposal device 1 of this kind, an accident whereby the object leaks out of the circular through-hole 5, the mouth section 7 and the disposal bag 6 occurs sometimes. It is therefore still pointed out that the device 1 has a disadvantage in that the subject adheres to the body or fingertips and gives out a bad odor.

Further, in the conventional excrement disposal device 1 of this kind, the front and rear frames 3 are adapted to open and close at an equal angle in the center to allow the right and left frames 2 to move in and out from right to left in parallel. However, it is often the case that the front and rear frames 3 do not open and close at an equal angle, the right and left frames 2 do not move in parallel as expected, and the mouth section 7 is not formed in such a substantially circular-ring shape as to match the shape of the anus and often deteriorates. Even from this aspect, it is pointed out that an accident in which the object leaks out as described above takes place.

### SUMMARY OF THE INVENTION

(Present Invention)

In view of these actual conditions, an excrement disposal device of the present invention was developed to solve the problems stated above as a result of the inventor's persistent and assiduous research efforts.

It is therefore an object of the present invention to provide an improved excrement disposal device in which (1) a mouth section can be correctly applied to the subanal area for positioning and (2) the operation of the device is stabilized to allow the mouth section to correctly open and close in accordance with the opening and closing motion of the anus.

### MEANS FOR SOLVING THE PROBLEMS

(Each aspect)

A technical means of the present invention for attaining such an object is described below.

(Aspect 1)

A handy-type excrement disposal device according to a first aspect is provided, which comprises a base plate, a wing plate and a disposal bag.

The base plate is provided in pairs (i.e., right and left base plates) and the front and rear ends of each plate are pivotally attached to each other to make the dimensions of an opening between the right and left base plates variable.

The wing plate is also provided in pairs (i.e., front and rear wing plates) to be mounted on both base plates, and a top section of each wing plate is opposedly situated to face the opening from ahead and from behind to control the front and rear shape of the opening, thereby making the size of a mouth section variable.

The disposal bag is vertically inserted into the mouth section formed by both wing plates. A mouth periphery of the inserted disposal bag can be caused to touch a subanal area from below for positioning and can be opened and closed in accordance with the opening and closing motion of the anus.

(Aspect 2)

The excrement disposal device according to the aspect 1 is provided, in which the right and left base plates are longitudinally formed in a substantially long-plate shape to be bent upwards, at least the front and rear ends of each plate are adapted to overlap to be pivotally attached to each other, and each plate is provided with a cutaway section in the center.

The opening is formed by at least the right and left cutaway sections. The dimensions of the opening are made variable based on the displacement of both plates in the lateral direction, using the pivot point of the front and rear ends as a fulcrum.

(Aspect 3)

The excrement disposal device according to the aspect 2 is provided, in which each base plate has a central area adjacent to the cutaway section bent upwards in advance in a substantially convex shape.

(Aspect 4)

The excrement disposal device according to the aspect 2 is provided, in which each base plate is provided with a cut-in tongue piece near the front and rear ends, respectively, and the tongue piece is adapted to sandwich a base end of the wing plate therebetween to allow the wing plate to slide along the base plate.

(Aspect 5)

The excrement disposal device according to the aspect 1 is provided, in which the front and rear wing plates can be slightly bent upwards to move back and forth while slidingly contacting each base plate, and one end of the base end section is pivotally attached to one of the right and left base plates, while the other end of the base end section is pivotally attached to another base plate.

The size of the mouth section is made variable by displacing both base plates in the right and left direction, using the pivot point of the front and rear ends as a fulcrum, and by allowing the top section of each wing plate to longitudinally come close or away in conjunction with the displacement of each base plate.

(Aspect 6)

The excrement disposal device according to the aspect 1 is provided, in which the disposal bag is detachably mounted on the excrement disposal device, an upper end section, of which the edge is open, is reversed to cover both base plates and wing plates, and a bottom section is provided to cause an object excreted from the anus to flow down through the mouth periphery for housing.

(Aspect 7)

The excrement disposal device according to the aspect 6 is provided, in which the disposal bag is made of a flexible resin material and a waist body is attached to the disposal bag from the mouth periphery toward the bottom section to lower the flexibility of the disposal bag in the area where the waist bag is attached and to facilitate the downward flow of the object.

(Aspect 8)

The excrement disposal device according to the aspect 1 is provided, in which the right and left spaces of the mouth section are provided to receive a fingertip, respectively, and the disposal bag of the mouth periphery controls an enclosure whereby the inserted fingertips do not project too far upward.

The mouth section is controlled between the inserted right and left fingertips and each top section of the front and rear wing plates to make both the lateral dimensions and the longitudinal dimensions variable, and the mouth periphery of the disposal bag can be formed in a substantially circular-ring shape to match the shape of the anus.

(Aspect 9)

The excrement disposal device according to the aspect 8 is provided, in which a finger-inserting section is respectively attached to the underside of a cutaway section of each base plate to cause the fingertip to be inserted therein from the bottom up and to cause the fingertips to be pushed against the anus from directly below through the disposal bag of the mouth periphery.

The finger-inserting section also laterally displaces the base plate by the movement of the inserted fingertips in the right and left direction according to the opening and closing motion of the anus, using the pivot point of the front and rear ends of each base plate as a fulcrum to make both the lateral size and the longitudinal size of the mouth section variable, thereby causing the disposal bag of the mouth periphery to open and close.

(Operation)

Operation of the present invention will now be described hereunder.
(1) In use of the excrement disposal device, the tips of the first finger and the second finger are first inserted into the right and left spaces of the mouth section respectively through each finger-inserting section. The excrement disposal device is held by two fingertips and is led to an area of the anus by slidingly moving it from the right and left side of a body, that is, from the side of a waist in a bent posture. In this case, the excrement disposal device is situated to allow the front and rear direction (i.e., longitudinal direction) to extend to the right and left direction of the body and allow its right and left direction (i.e., lateral direction) to extend to the front and rear direction of the body.
(2) The excrement disposal device is thus applied to the anus. Namely, the front and rear ends of each base plate are extended to the right and left direction of the human body, while the right and left spaces of the mouth section are directed to the front and rear direction of the human body, wherein the fingertips are applied to the front and rear area of the anus from directly below.
(3) In this manner, the excrement disposal device is correctly applied to the subanal area of the anus. Namely, two fingertips inserted into the right and left spaces of the mouth section properly grasp, identify and confirm the location of the anus and are pushed against it.
(4) The two fingertips are first caused to approach to close, and the mouth periphery of the disposal bag is closed.
(5) After evacuation, an object is caused to flow down and is housed in the disposal bag of the excrement disposal device. This can be carried out only by leaving the opening and closing of the anus entirely to the two fingertips pushed against the subanal area through the mouth periphery of the disposal bag.
(6) Namely, when the anus is opened or closed according to the evacuation of the object, the two fingertips also symmetrically come close or come away to open or close, and the mouth section is opened or closed while horizontally or vertically changing the size through the finger-inserting sections, base plates and wing plates which symmetrically come close or recede. The mouth periphery of the disposal bag is therefore formed in a substantially circular-ring shape or in a closed condition according to the shape of the opening or closing anus.
(7) The opening and closing operation of the mouth periphery of the disposal bag can be more stably secured and guaranteed by factors such as overlapping of the base plates, bending and tongue pieces of the base plates, attachment of the mouth periphery of the disposal bag, and positioning of the fingertips playing an important role in opening and closing in the center of the excrement disposal device.

The mouth section of the excrement disposal device is formed by the base plates and the wing plates, but the base plate is deformed between the longitudinally long closed condition and the expanded condition of a substantially truncated cone shape in which the base plates approach each other from the longitudinal direction to rise. The wing plates are also deformed according to the deformation of the base plates. With this, the mouth periphery of the disposal bag opens or closes while keeping the same height on the same level and as a result, a displacement or gap is not caused relative to the opening or closing anus.
(8) By continuing to push the two fingertips against the anus from directly below through the mouth periphery of the disposal bag, an edge of the top section of each wing plate also continues to be pushed against the anus from directly below. Even by such pushing from the lateral and vertical directions, it is possible to prevent a displacement or gap from being produced between the mouth periphery of the disposal bag and the anus and to cope with the vertical motion of the anus.
(9) After excretion, the anus is closed and the two fingertips pushed against the anus also approach to a closed position, whereby the mouth periphery of the disposal bag is brought to a closed condition. The upper end section of the disposal bag is then returned to the extended condition from the folded (reversed) condition, whereby the upper edge of the disposal bag is closed by a string operation and the mouth periphery is tied with a string from the outside.
(10) The disposal bag in which the object is housed is removed from the excrement disposal bag and is housed in an outer bag. The outer bag is then sealed off to be scrapped. The disposal bag can also be thrown away together with the excrement disposal device after use. In other words, the excrement disposal bag with the disposal bag can be housed in the outer bag and be sealed off to be scrapped.

### BRIEF DESCRIPTION OF THE DRAWINGS

The above and other objects, features and advantages of the present invention will become more apparent from the following description when taken in conjunction with the accompanying drawings.

FIG. 1 is an expanded bottom view of an excrement disposal device according to the present invention in use for describing the best mode for carrying out the present invention;

FIG. 2 is provided to describe the best mode for carrying out the present invention, wherein FIG. 2A is a closed top view of the excrement disposal device in use and FIG. 2B is an opened top view of the excrement disposal device in use;

FIG. 3 is a side view of the excrement disposal device in use with the substantial part cutaway, for describing the best mode for carrying out the present invention;

FIG. 4 shows each use step of the excrement disposal device for describing the best mode for carrying out the present invention, wherein FIG. 4A is a side view of the excrement disposal device in use, FIG. 4B is a rear view of the device in use, and FIG. 4C is an enlarged view showing the substantial part of FIG. 4B. FIGS. 4D through 4G are schematic front views showing each step after use in order;

FIG. 5 is a top view of a base plate for describing the best mode for carrying out the present invention, wherein FIG. 5A shows a condition of the base plate before being pivotally attached, FIG. 5B shows a condition in which the base plate is pivotally attached and the size of an opening is small, and FIG. 5C shows a condition in which the size of the opening is large;

FIG. 6 is a top view of a wing plate mounted on the base plate to describe the best mode for carrying out the present invention, wherein FIG. 6A shows a condition in which the size of a mouth section is small and FIG. 6B shows a condition in which the size of the mouth section is large. FIG. 6C shows an example in which the base plate is provided with a tongue piece;

FIG. 7 shows a finger-inserting section attached to the underside of the base plate to describe the best mode for carrying out the present invention, wherein FIG. 7A is a bottom view of the finger-inserting section in a condition before the two base plates are pivotally attached, FIG. 7B is a bottom view of the finer-inserting section in a closed condition in which two base plates are pivotally attached to each other, and FIG. 7C is a side view of the finger-inserting section; and

FIG. 8 is a perspective view of a conventional excrement disposal device of this kind.

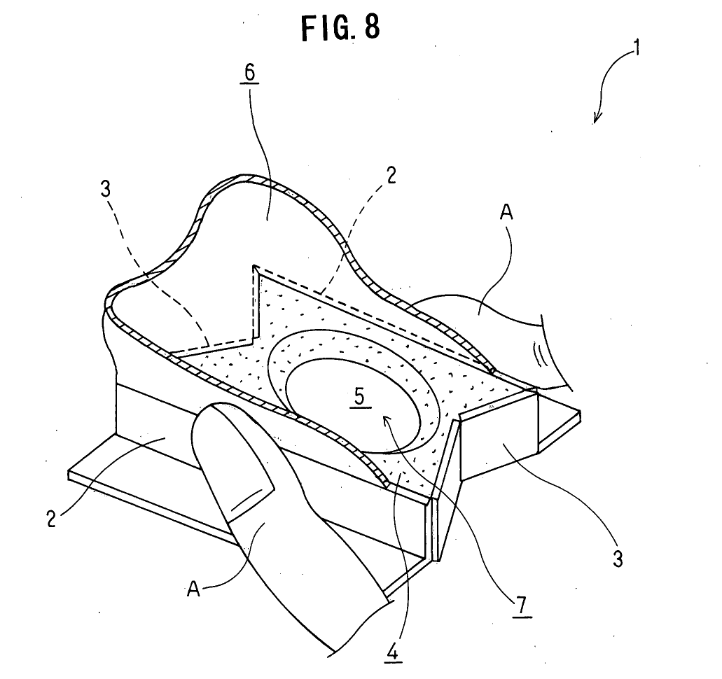

### DETAILED DESCRIPTION OF THE PREFERRED EMBODIMENTS

(Outline of the Excrement Disposal Device 8)

An outline of the excrement disposal device 8 according to the present invention will now be described. This excrement disposal device 8 is made portable for use in outdoor activities, nursing-care services, military services or the like. This can be widely used as a handy-type excrement disposal device when toilet facilities are not available in the open air, be used for nursing-care services for a bed-ridden person on a bed, for use within a military vehicle at the battlefront and the like.

The excrement disposal device 8 has a base plate 9, a wing plate 10, a finger-inserting section 11, a disposal bag 12 and the like and is intended for an object such as excremental matter from a human body (a typical example is excrement, but urine and vomit are also acceptable).

The base plate 9, the wing plate 10 and the finger-inserting section 11 are made of a polypropylene sheet material, a resin sheet, a paper sheet or the like with bendability (flexural properties) of about 0.75 mm thickness which can be die-cut and then, slit up and bent, if necessary. The disposal bag 12 is made of a polyethylene film or other resin film which is flexible and is colored so that the content can not be seen.

Detailed description of the base plate 9, the wing plate 10, the finger-inserting section 11 and the disposal bag 12 are given hereunder.

(Base Plate 9)

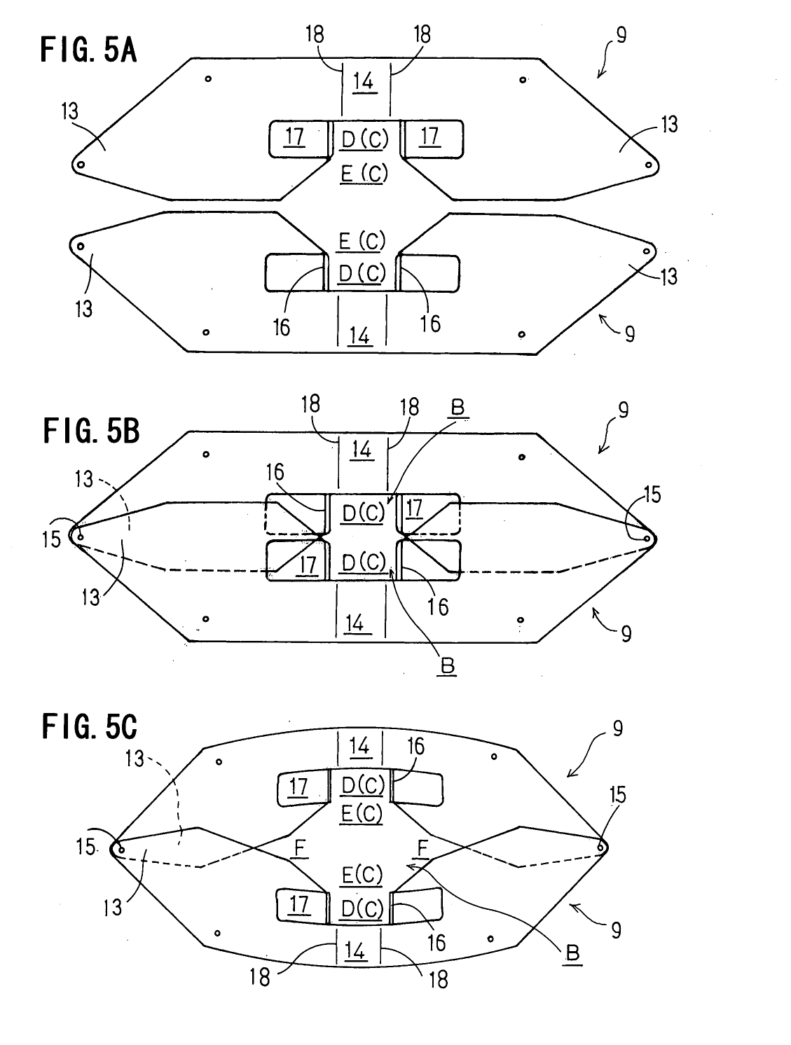

The base plate 9 of the excrement disposal device 8 will be described with reference to FIG. 5 and other relevant figures. The base plate 9 is provided in pairs (i.e., right and left base plates). The front end 13 and the rear end 13 of each plate 9 are pivotally attached to each other and the size of an opening B is made variable between the right and left base plates 9 and 9.

Each base plate 9 is longitudinally formed in a substantially long plate shape and can be bent upwards. In an example as shown in FIG. 5, the front end 13 and the rear end 13 overlap to be pivotally attached to each other and each base plate is provided with a cutaway section C in the center. The opening B is formed by at least the right and left cutaway sections C and the size of the opening B is made variable based on the symmetric displacement of the right and left base plates 9 in the right and left direction, using the pivot point of the front end 13 and the rear end 13 as a fulcrum.

The base plate 9 of about 140-150 mm long and 35 mm wide is longitudinally formed in a substantially long-plate shape to make the central section 14 high and to make the front end 13 and the rear end 13 (hereinafter referred to as “front and rear ends”) low. The base plate 9 can be bent upwards.

In the examples as shown in FIG. 5 and other figures, a pair of base plates 9 shown in FIG. 5A is provided to cause the front and rear ends 13, 13 to partially overlap, as shown in FIGS. 5B and 5C, and the front and rear ends 13, 13 are rotatably secured in a two-dimensional manner, making a pin 15 of the vertical axis a pivot point. The overlapping width is about 20 mm in a condition of FIG. 5B.

The base plates 9 can be displaced between a longitudinally extended condition as shown in FIG. 5B and a substantially truncated cone shape, that is, a reverse cone or bowl shape, as shown in FIG. 5C, in which the two base plates 9, 9 approach to make the longitudinal direction short, thereby expanding in the lateral direction to rise as a whole, using the pin 15 as a fulcrum.

The pivot pin 15 is normally a separate member from the base plates 9, but can be made integrally with the plates 9. In such a case, the pin is integrally set between the base plates 9 from the initial stage of molding.

Regardless of the examples as shown in FIG. 5 and other figures, it is also possible to cause the front and rear ends 13 of the right and left base plates 9 not to overlap at all so that the base plates 9 are installed side by side. In this case, the pin 15 must be a pin of a horizontal axis. In a typical example of this type, one flat plate is provided with a slit in the center to form a pair of base plates 9. An edge of the front and rear ends 13 of such base plates 9 is not provided with a slit to leave the area as is and this integrally connected area is used as a pin 15 of a horizontal axis.

The inner side of each base plate 9 in the center is symmetrically provided with a cutaway section C opening inside. One continuous opening B is formed by the right and left cutaway sections C between two base plates 9.

The size of the opening C is made variable. That is, the size of the opening B is made large or small based on the symmetric displacement of both base plates 9 in the right and left direction (by) using the pin 15 serving as the pivot point of the front and rear ends 13 as a fulcrum. The cutaway section C as shown in the examples of FIG. 5 and other relevant figures is continuously provided with right and left areas D forming a rectangular shape at the back and with trapezoidal areas E with a slope on the inside, the mutually corresponding side and the open side.

In FIG. 5B, only the right and left areas D are exposed (in a condition without the disposal bag 12) to make the size of the opening B minimal. In FIG. 5C, the right and left areas D, the right and left trapezoidal areas E, and a central area F formed between the trapezoidal areas E and by the inner sides of both base plates 9 are exposed (in a condition without the disposal bag 12) to make the size of the opening B maximal.

Each base plate 9 as shown in the examples of FIG. 5 etc. has two (front and rear) guide projections 16, situated adjacent to each other on the front and rear sides of the right and left areas D of the cutaway section C, which droop at right angles. These front and rear guide projections 16 are formed by folding a remaining side of a hole 17, of which the three sides are cut in, to vertically droop and serves as a guide for the fingertips A.

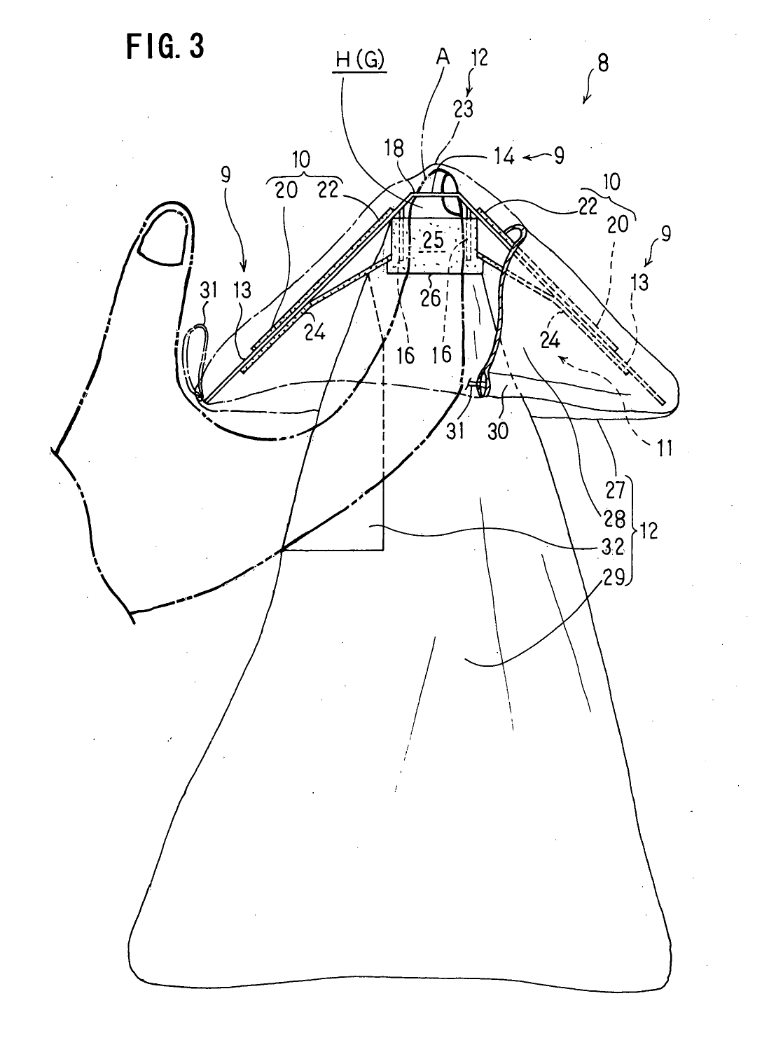

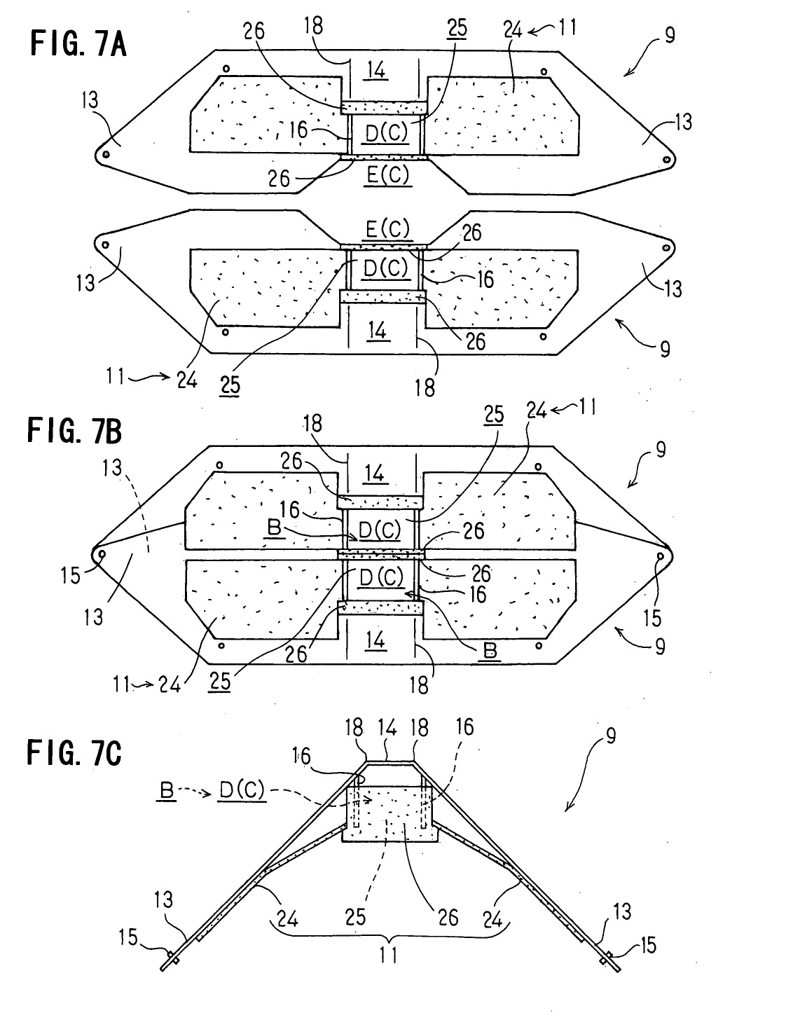

Each base plate 9 as shown in the example is also provided with a central section 14 situated adjacent to the right and left areas D of the cutaway section C. The central section 14 is formed by two bent sections 18 bent upwards in a substantially convex shape. These bent sections 18 are formed by creasing the front and rear ends of the central section which are separated by about 10 mm. In this manner, as shown in FIGS. 3 and 7C, the base plate 9 is provided so that the front and rear ends 13 and 13 are at an angle of about 90 degrees.

These bent sections 18 function to reduce the load on the fingertips A being basic to the displacement motion in the case where the base plates are caused to displace in the right and left direction while being bent, using the pin 15 which is a pivot point of the front and rear ends 13 and 13 pivotally attached as a fulcrum. In other words, the bent sections 18 function to stably displace both base plates 9.

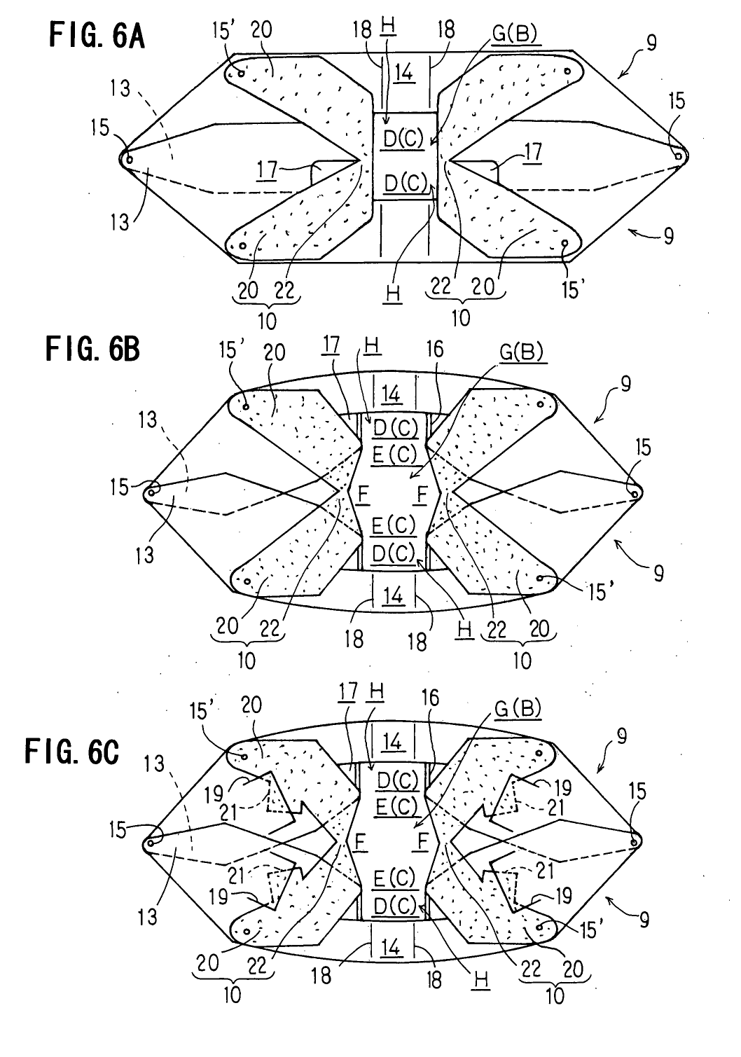

Further, referring to an example as shown in FIG. 6C, each base plate 9 is provided with two cut-in tongue pieces 19, near the front and rear ends 13 and 13 thereof, adapted to sandwich each base end 20 of the front and rear wing plates 10 therebetween. The tongue piece 19 serves to slide the wing plate 10 along the base plate 9.

Namely, each base plate 9 as shown in the example of FIG. 6C is respectively provided with two cut-in tongue pieces 19 of a substantially horseshoe-shape near the front and rear ends 13 and 13, while a guide projection 21 is projectingly provided on the inside of each forked base end section 20 of the wing plate 10 to correspond to each tongue piece 19. In this excrement disposal device, 4 pieces each of the tongue pieces 19 and the guide projections 21 are provided.

The tongue piece 19 is respectively provided to put the guide projection 21 thereunder and guides to allow the wing plate 10 to slide on the base plate 9. Namely, the tongue piece 19 functions to stably slide the wing plate 10 so that the top section 22 of the sliding wing plate 10 does not warp upwards. In particular, the tongue piece 19 guides so as not to cause the top section 22 of the wing plate 10 to warp to produce a step when each wing plate 10 slides from the receded condition to the close condition to move back and forth.

(Wing Plate 10)

The wing plate 10 of this excrement disposal device 8 will be described hereunder with reference to FIG. 6 and other figures. The wing plate 10 is provided in pairs (i.e., front and rear wing plates) to be mounted on the base plate 9. The top section 22 of each wing plate 10 is opposedly situated to face an opening B, from ahead and from behind, formed between the base plates 9 and controls the shape of the opening B in the front and rear direction to make the size of a mouth section G variable.

The front and rear wing plates 10 can be slightly bent upwards to symmetrically move back and forth and is provided to slide on both base plates 9.

The wing plate 10 is provided so that one end of the base end section 20 is pivotally secured to one of the right and left base plates 9, while another end of the base end section 20 is pivotally secured to another base plate 9. The size of the mouth section G is made variable by the displacement of the two base plates 9 in the horizontal directions, using the pivot pin 15 of the front and rear ends 13 and 13 as a fulcrum, and by the motion of longitudinally approaching or receding from the top sections 22 of both wing plates 10 in conjunction wit the displacement.

Such a wing plate 10 is further described in detail. Each wing plate 10 consists of the top section 22 and the forked base end section 20. The top section 22 is forked horizontally through a narrow central edge of, for example, about 5 mm width in the longitudinal direction, while the base end section 20 is provided as a continuation of the right and left forked section of the top section 22 to have a substantially fork shape.

The top section 22 is provided with displacement characteristics, folding characteristics, and bending characteristics in the center of the edge. The right and left base end sections 20 are pivotally secured to the corresponding right and left base plates 9, respectively, in a two-dimensional manner by pins 15′ of a vertical axis in the vicinity of the end. The distance between the right and left pins 15′ and 15′ is about 40 mm.

The pivot pin 15′ is normally made of a different material from the wing plate 10 and the base plate 9, but can be integrally formed with the base end section 20 of the wing plate 10 and the base plate 9. In this case, the pin 15′ is integrally set between the base end section 20 and the base plate 9 from the initial stage of molding.

The wing plates 10 are opposedly situated to be symmetrical in the longitudinal direction, facing the opening B from ahead and from behind and control the shape of the opening B in the longitudinal direction to form the mouth section G.

When the two base plates 9 are symmetrically displaced in the horizontal direction, the two wing plates 10 are also symmetrically displaced in the longitudinal direction through a pivot point by the pin 15′. With this displacement, the mouth section G formed based on the opening B is made variable by engaging the lateral dimensions with the vertical dimensions.

In other words, the two wing plates 10 can be displaced between the condition in which the edge of the top section 22 is substantially straight, as shown in FIG. 6A and the condition in which the edge of the top section 22 is bent to make the central section high and to make the right and left sections low, and is bent in a substantially V-shape to allow the right and left sections to approach, as shown in FIGS. 6B and 6C.

The displacement of such wing plates 10 results from the fact that the base plates 9 are displaced between the condition as shown in FIG. 5B and the condition as shown in FIG. 5C, as described above, and the wing plates 10 adapted to slide on the base plates 9 are also displaced. A substantially circular-ring shape of a mouth periphery 23 of the disposal bag 12 described below is accelerated whenever the wing plates 10 are displaced under the conditions shown in FIGS. 6B and 6C.

In the case where the size of the opening B formed by the two base plates 9 is small (refer to FIG. 5B), the dimensions of the mouth section G based on the opening B and controlled by the wing plates 10 also becomes small (refer to FIG. 6A). On the contrary, in the case where the dimensions of the opening B is large (refer to FIG. 5C), the dimensions of the mouth section G also becomes large (refer to FIGS. 6B and 6C).

Each edge of the top sections 22 of the front and rear wing plates 10 is provided with a cushion piece (not shown) made of, for example, rubber. This cushion piece is caused to touch the anus area from below through the mouth periphery 23 of the disposal bag 12 to exhibit the cushion performance against the anus and the eradicative function for the vertical clearance between the anus and the cushion piece.

(Finger-Inserting Section 11)

The finger-inserting section 11 of this excrement disposal device 8 will now be described with reference to FIG. 7 and other relevant figures. The finger-inserting section 11 is provided in pairs (i.e., the right and left finger-inserting sections) and is attached to the underside of the cutaway section C of each base plate 9 to cause the fingertip A to be inserted therein from the bottom up. The finger-inserting section 11 also guides the inserted fingertip A to cause it to be pushed against the anus area from directly below through the mouth periphery 23 of the disposal bag 12.

Further, the finger-inserting sections 11 laterally displace the two base plates 9 by the movement of the fingertips A, adapted to move in conjunction with the opening and closing motion of the anus, in the horizontal direction, using a pin 15 serving as a pivot pin of the front and rear ends 13, 13 as a fulcrum to make the horizontal dimensions and the vertical dimensions of the mouth section G variable, thereby causing the mouth periphery 23 of the disposal bag 12 to open or close.

The finger-inserting section 11 will now be further described in detail. The finger-inserting section 11 is used in pairs (i.e., right and left finger-inserting sections) to be mounted on the right and left base plates 9. In an example as shown in FIG. 7, the front and rear extended plate sections 24 of each finger-inserting section 11 are fixedly secured to the base plates by, for example, a stapler.

Each finger-inserting section 11 is provided with a finger-inserting hole 25 in the center. This finger-inserting hole 25 is formed in a longitudinally long rectangular shape by cutting the front and rear sections and the central section in a substantially I-shape and horizontal H-shape, respectively and laterally bending those sections to droop. A pair of right and left guide projections 26, 26 is formed by bending those sections to droop and function as a guide for a fingertip A.

The finger-inserting hole 25 of each finger-inserting section 11 is opposedly situated on the underside of the right and left areas D of the cutaway section C of each base plate 9. The right and left edges of the finger-inserting hole 25 are provided with right and left guide projections 26 to be drooped downwards. Inserted into the front and rear edges of the finger-inserting hole 25 to be drooped are the front and rear guide projections 16 as described above.

In this finger-inserting section 11, the fingertip A can be inserted into the finger-inserting hole 25 from the bottom up while being guided by the right and left guide projections 26 and the front and rear guide projections 16. The fingertip A can also be inserted into the right and left areas D of the cutaway section C, the corresponding area of the opening B and the right and left spaces H of the corresponding mouth section G, and can be directed to the mouth periphery 23 of the disposal bag 12.

In this manner, the finger-inserting section 11 can be moved in the horizontal direction by the inserted fingertips A to cause the right and left base plates 9, to which both finger-inserting sections 11 are fixedly secured, to symmetrically displace. With this, the lateral dimension and the vertical dimension of the mouth section G thereof are made variable to open or close the mouth periphery 23 of the disposal bag 12.

For example, when the fingertips A of the right and left finger-inserting sections 11 laterally move inside to come closer, the right and left base plates 9 and the front and rear wing plates 10 also come close to make the size of the opening B small, thereby making the size of the mouth section G small. To the contrary, in the case where the fingertips A of the right and left finer-inserting sections 11 laterally move outside to separate, the right and left base plates 9 and the front and rear wing plates 10 also separate to make the size of the opening B large, thereby making the size of the mouth section G large.

FIG. 7B shows the condition in which the size of the mouth section G is minimal (this corresponds to FIGS. 5B and 6A described above). In this condition, the right and left guide projections 26 of the right and left finger-inserting sections 11 are caused to touch each other from the horizontal direction, wherein the fingertips A inserted into the right and left finger-inserting sections 11 are also caused to substantially touch each other from the horizontal direction through the mouth periphery 23 of the disposal bag 12.

It is to be noted that the inner corner of one extended plate section 24 of the right and left finger-inserting sections 11 is designed to slightly warp downwards. With this design, when the inner corner of another corresponding extended plate section 24 moves closer, it slips into the inner corner of one non-corresponding extended plate section warped not to collide.

(Disposal Bag 12)

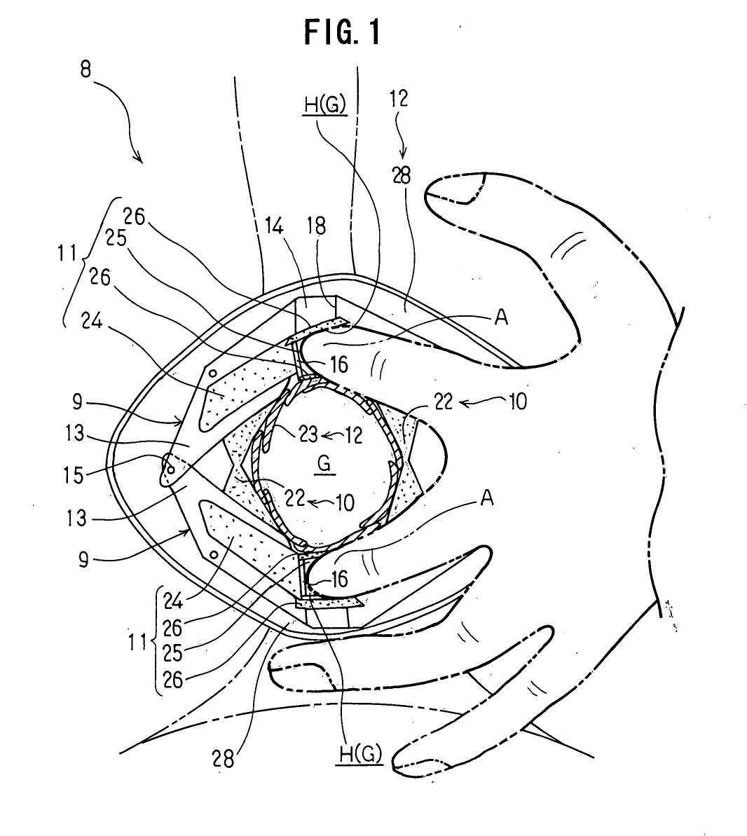

The disposal bag 12 of this excrement disposal device 8 will now be described with reference to FIGS. 1, 2, 3 and other relevant figures. The disposal bag 12 is vertically inserted into the mouth section G formed by both wing plates 10 and the like to allow the mouth periphery 23 to touch a subanal area from below for positioning. The mouth periphery 23 is adapted to open and close according to the opening and closing motion of the anus.

Namely, the disposal bag 12 is detachably and additionally provided, wherein an upper end section 28 of which the edge 27 is open is reversed to cover both base plates 9 and wing plates 10, and a bottom section 29 is adapted to receive an object excreted from the anus and house it.

Fingertips A can be inserted into the right and left spaces H of the mouth section G, respectively. The mouth periphery 23 of the disposal bag 12 is provided to control an enclosure which prevents the inserted fingertips A from projecting upwards to too great an extent. The shape of the mouth section G formed based on the opening B is controlled to make both the lateral dimensions and the vertical dimensions variable between the right and left fingertips A and the top sections 22 of the front and rear wing plates 10, wherein the mouth periphery 23 of the disposal bag 12 is formed in a substantially circular-ring shape and the like to match the shape of the opening and closing anus.

The disposal bag 12 is further described below in detail. The disposal bag 12 is formed in a pouched shape of about 350 mm high and about 180 mm wide and consists of the upper end section 28, the mouth periphery 23 and the bottom section 29. The bag 12 is always used in a substantially flat condition in which air is removed.

The upper end section 28 is provided with a hollow string passage 30 for lacing a string 31 around the edge 27. The disposal bag 12 is opened and closed by the operation of the string 31. The bag 12 is opened before and when used as the excrement disposal device 8 and is closed after use.

After the disposal bag 12 is vertically inserted into the mouth section G, the upper end section 28 is reversed to cover the base plates 9 and the wing plates 10.

The mouth periphery 23 of the disposal bag 12 not only includes a section vertically inserted into the mouth section G, but also an opening flat surface section adjacent to the vertically inserted section.

The right and left spaces H of the mouth section G are adapted to receive the two fingertips A respectively through the finger-inserting holes 25 of the finger-inserting sections 11, the right and left areas D of the cutaway sections C, the corresponding areas of the opening B, and the like. The vertical and horizontal size (shape) of the mouth section G formed based on the opening B is controlled by the inserted right and left fingertips A and the front and rear wing plates 10.

Since the disposal bag 12 of a larger diameter is inserted into the mouth section G of which the shape is controlled, and is reversed, the mouth periphery 23 of the disposal bag 12 is adapted to fit into the mouth section G and each corner section is additionally provided with a curvature to provide large overlapping wrinkles. Thus, the mouth periphery 23 of the disposal bag 12 is formed in a substantially circular-ring shape in many cases based on the curvature and the overlapping wrinkles on each corner.

Further, the size of the mouth periphery 23 of such a disposal bag 12 is made variable according to the opening and closing shape of the anus and the size change of the mouth section G.

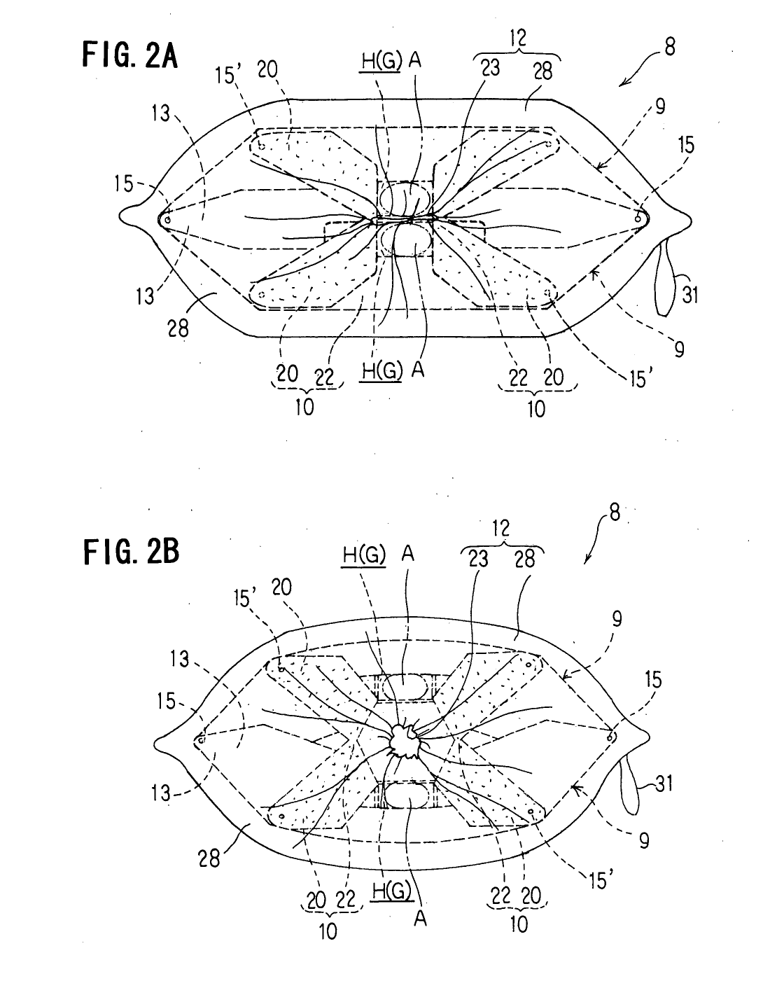

More specifically, in the closed condition as shown in FIG. 2A, the right and left guide projections 26 of the finger-inserting sections 11 touch each other from the right and left directions and the two fingertips A through the finger-inserting sections 11 come close to such a degree that these fingertips A touch each other through the right and left areas D of the cutaway sections C and the right and left spaces H of the mouth section G (These correspond to FIGS. 5B, 6A and 7B).

In this case, the mouth periphery 23 of the disposal bag 12 becomes a minimum size in a substantially closed condition. Namely, the mouth periphery 23 becomes a groove-shape close to a vertical linear shape to correspond to the closed anus rather than a substantially circular-ring shape.

On the contrary, in the expanded condition as shown in FIGS. 1 and 2B, both fingertips A of the right and left spaces H of the mouth section G are laterally expanded. Referring to the example of FIGS. 1 and 2B, the mouth section G of a polygonal shape such as a substantially hexagonal shape and a substantially decagonal shape is formed by the trapezoidal area E of the cutaway sections C, the central area F, the top sections 22 of the front and rear wing plates 10, and the like (these correspond to FIGS. 5C, 6B and 6C).

In this case, the mouth periphery 23 of the disposal bag 12 is formed in a substantially circular-ring shape to match the shape of the anus at that time, but this substantially circular-ring shape can be changed to various sizes depending on the opening and closing degree of the anus and the size change of the mouth section G.

The mouth periphery 23 of such a disposal bag 12 is affixed to the finger-inserting sections 11, the base plates 9 and the wing plates 10, respectively with, for example, a double-faced adhesive tape.

More specifically, the outer surface of the mouth periphery 23 of the disposal bag 12 is secured to one vertical surface of the right and left guide projections 26 of each finger-inserting section 11 near the center, the upper surface of the central section 14 of each base plate 9, and the edge of the top section 22 of each wing plate 10 (i.e., an area of the cushion piece described above) at, for example, six (6) locations, with the double-faced adhesive tape.

The mouth periphery 23 of the disposal bag 12 secured at these 6 locations is provided to surround and cover the fingertips A which are inserted into the right and left spaces H of the mouth section G to project and to control so that the fingertips A do not project further upward. Thus, the mouth periphery 23 of the disposal bag 12, secured in 6 locations, can stably be formed in a substantially circular-ring shape.

The bottom section 29 of the disposal bag 12 causes the object to enter through the mouth periphery 23 to be housed. As shown in FIG. 3, a waist body 32 is attached to the disposal bag 12 from its mouth periphery 23 toward the bottom section 29 to reduce the flexibility of the attached area, thereby facilitating the transfer of the object.

The waist body 32 is preferably made of a firm tape and the like which are harder than the resin film material etc. used in the disposal bag 12 and is affixed to the outer surface of the disposal bag 12 from the lower area of the mouth periphery 23 toward the bottom section 29. The waist body 32 functions to smoothly let an object, especially a soft object, flow from the mouth periphery 23 toward the bottom section 29.

(Operation and the Like)

The excrement disposal device 8 according to the present invention is constructed as described above and is used as described below.

(1) In use, the excrement disposal device 8 is first put on the palm of the left hand letting the string 31 face the front. The fingertip A of the first finger and the fingertip A of the second finger of the right hand are respectively inserted into the right and left spaces H of the mouth section G from below through the right and left finger-inserting sections 11 (This applies to a right-handed person. In the case of a left-hander, the right and left hands are reversed).

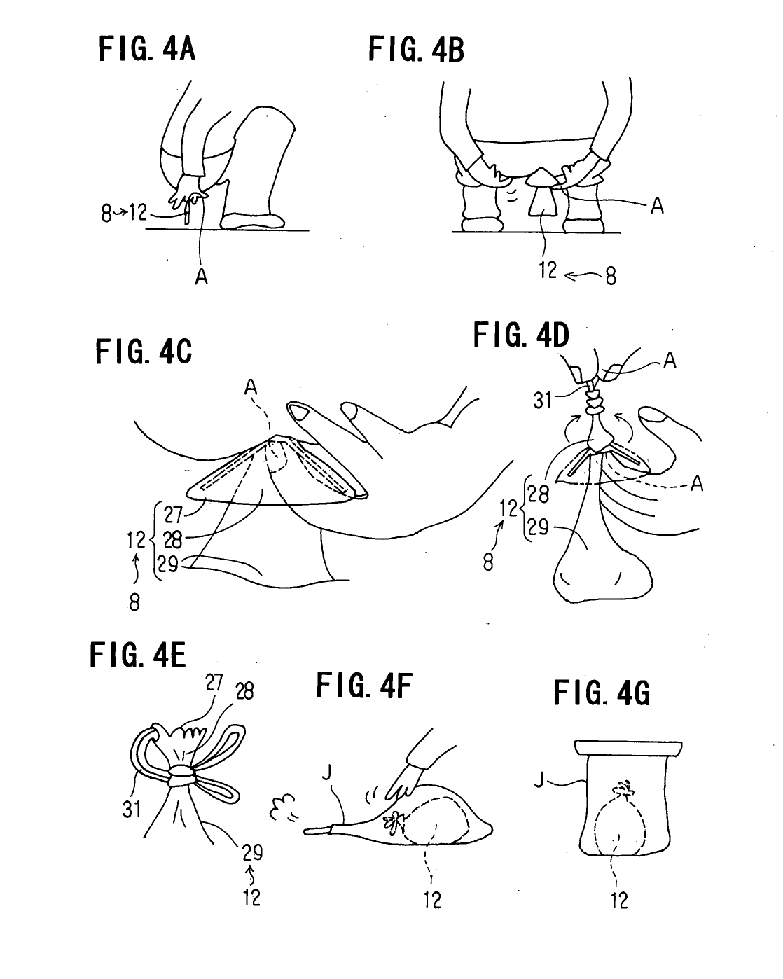

As shown in FIG. 4A, the excrement disposal device 8 of which the mouth section G is directed upwards is held by two fingertips A of the right hand. The hand held out from the side of a human body bent down, that is, from the hip side, is then moved to guide the fingertips A and the excrement disposal device 8 sliding on the swelling of the buttocks to the anus area.

In this case, the excrement disposal device 8 is provided so that the front and rear direction (i.e., the longitudinal direction) of the device 8 is directed to the right and left direction of the human body, while the right and left direction (i.e., the short direction) of the device 8 is directed to the front and rear direction of the human body.

(2) In this manner, the excrement disposal device 8 is applied to a subanal area from directly below. More specifically, as shown in FIGS. 4b and 4C, the excrement disposal device 8 is provided, in which the front and rear ends 13 and 13 of each base plate 9 are directed to the right and left direction of the human body to move along the swelling of the buttocks, while the right and left spaces H of the mouth section G are directed to the front and rear direction of the human body to move along the reverse cleavage.

The excrement disposal device 8 is directed in this manner and the two fingertips A, A holding the excrement disposal device 8 are applied to the front and rear areas of the anus from directly below. The mouth section G is adapted to open and close depending on the subsequent approaching movement or receding movement of the two fingertips A.

(In the following description, the right and left direction of the excrement disposal device 8 including the fingertips A is the front and rear direction of the human body, and the front and rear direction of the device 8 including the fingertips A is the right and left direction of the human body.)

(3) As shown in FIGS. 4B and 4C, the excrement disposal device 8 is correctly applied onto the subanal area for positioning.

In other words, both fingertips A inserted into the right and left spaces H of the mouth section G from directly below can correctly grasp and identify the location of the anus from directly below through the mouth periphery 23 of the disposal bag 12. Both fingertips A grope for and confirm the location of the anus and are pushed against the subanal area after positioning through the mouth periphery 23 of the disposal bag 12.

In this manner, based on direct feel, that is, the tactile impression, of the fingertips A relative to the anus, the fingertips A can be correctly brought into line with the center of the mouth section G of the excrement disposal device 8, the center of the mouth periphery 23 of the disposal bag 12, and the center of the anus for proper positioning without causing any displacement and gap. Thus, the object is caused to enter the disposal bag 12 through the mouth periphery 23 and is collected as expected.

(4) In the excrement disposal device 8 applied onto the subanal area in this manner, the inserted two fingertips A, A are first caused to approach from the side to such a degree that they touch each other, as shown in FIG. 2A and other figures, and are closed. The mouth periphery 23 of the disposal bag 12 is closed.

If an attempt to open the anus which becomes narrower is made with a free hand, the confirmation, identification and positioning of the anus position by the fingertips A, A of the hand which is involved in the operation of the excrement disposal device 8 described above can be facilitated further. The touch and approach of the fingertips A, A described above can also be highly facilitated. If the mouth periphery 23 of the disposal bag 12 of the excrement disposal device 8 and the fingertips A, A can be pushed into (the inside of) the anus thus opened, the positioning described above can be further ensured.

In this manner, if the positioning of the excrement disposal device 8 relative to the anus is correctly carried out, the sense of unity between the anus, which becomes narrower, and the fingertips A, A can be created.

(5) After being excreted, the object from the anus which selectively opens or closes flows to the bottom section 28 of the disposal bag 12 through the mouth periphery 23 of the disposal bag 12 of the excrement disposal device 8 and is housed.

Housing of the object by such an excrement disposal device 8 can be readily and surely carried out by leaving the opening and closing of the anus entirely to the two fingertips A pushed against the subanal area from directly below through the mouth periphery 23 of the disposal bag 12.

In the case of excretion, although the anus opens and closes, changing the width, depending on the diameter of the object, the excrement disposal device 8 can surely help facilitate proper housing of the object.

(6) In this excrement disposal device 8, the mouth section G formed based on the opening B is controlled to make the size thereof variable by the right and left (two) fingertips A pushed against the anus area and the front and rear wing plates 10. Thus, the mouth periphery 23 of the disposal bag 12 can be formed in a substantially circular-ring shape to match the shape of the anus.

When the anus opens or closes in conjunction with the excretion of the object, the two fingertips A, A playing an important role in the opening and closing motion of the anus open or close moving to symmetrically and laterally approach or separate. Based on this, the mouth section G is opened or closed, making the size variable depending on the motion of the anus, through each finger-inserting section 11, each base plate 9, each wing plate 10 and the like which symmetrically approach or separate in the longitudinal and lateral directions. In other words, the dimensions of the mouth section G are made variable by controlling the opening B formed between the two base plates 9 in the longitudinal and lateral directions between the inner side of the fingertips A and the top section 22 of the wing plates 10.

In this manner, the mouth periphery 23 of the disposal bag 12 is stably and surely formed in the expanded condition of a substantially circular-ring shape of varying size corresponding to the shape of the anus as shown in FIGS. 1 and 2B, and in the closed condition corresponding to the shape of the closed anus as shown in FIG. 2A.

The mouth periphery 23 of the disposal bag 12 moves depending on the diameter of the anus sequentially changing, that is, opening and closing of the anus in the width, without displacement and gap, and is opened or closed to overlap matching the diameter of the opening anus. As a result, the object flows into the disposal bag 12 through the mouth periphery 23 as expected and is collected.

(7) The opening and closing operation of the mouth periphery, 23 of such a disposal bag 12 can be more stably secured, guaranteed and facilitated by the overlapping of the right and left base plates 9, the existence of the bent section 18 formed on each base plate 9, the existence of the tongue pieces 19, fixation of the mouth periphery 23 of the disposal bag 12 with the double-faced adhesive tape and the like as described above. Further, positioning of the fingertips A, A, playing an important role in the opening and closing operation, in the center of the excrement disposal device 8 and in the center of the base plates 9 is also a factor contributing to the stable operation.

Also, opening and closing of the mouth periphery 23 of such a disposal bag 12 is carried out with curvature. Even from this aspect, a substantially circular-ring shape can be formed to match the shape of the anus which opens and closes.

Namely, the mouth section G is formed by the opening B of the base plates 9 and the wing plates 10, but these base plates 9 and wing plates 10 are adapted to move in the longitudinal direction or in the lateral direction to approach or separate while being bent. More specifically, the base plates 9 are provided to totally deform between the closed condition as shown in FIG. 2A, that is, the long condition, and the expanded condition as shown in FIGS. 1 and 2B, that is, a substantially truncated cone-shape condition in which the base plates become short to approach and rise as a whole. The wing plates 10 also deform in accordance with the deformation of the base plates.

With this arrangement, the mouth periphery 23 of the disposal bag 12 is provided to open and close keeping the same height on the same level. Thus, the mouth periphery 23 is adapted to surely match the anus which also opens and closes on the same plane surface and on the same height level without causing any displacement and gap. Even from this aspect, a substantially circular-ring shape can be formed to match the shape of the anus.

(8) By continuing to push the two fingertips A against the anus area from directly below through the mouth periphery 23 of the disposal bag 12, the edge of the top section 22 of the wing plates 10 through the finger-inserting sections 11 and the base plates 9 can also be continuously pushed against the anus area from directly below through the mouth periphery 23 of the disposal bag 12.

By such pushing from the vertical and lateral directions, it is possible to prevent a displacement and a gap from being caused between the mouth periphery 23 of the disposal bag 12 of a substantially circular-ring shape matching the shape of the anus and the anus.

Further, with this pushing, it is possible to readily cope with the vertical movement of the anus. Namely, in the case where the object is not soft, the anus often moves vertically. Even in that case, it is possible to surely prevent the displacement and a gap from being caused between the mouth periphery 23 of the disposal bag 12 and the anus by such pushing.

(9) When the excretion is over in this manner, the anus is closed, and the right and left fingertips A, A pushed against the anus also approach to such a degree that the finger cushions substantially touch each other and are closed, as shown in FIG. 2A and other figures.

By strongly closing the two fingertips A, A, the remaining object adhering to the anus area is grasped and housed in the disposal bag 12, and the mouth periphery 23 of the disposal bag 12 is also closed.

In other words, after the right and left fingertips A, A are respectively inserted into the finger-inserting holes 25 of the finger-inserting sections 11, the mouth periphery 23 of the disposal bag 12 is only provided between the two fingertips A, A. Namely, since nothing hard is interposed between them, the two fingertips A, A can be closed after closely approaching each other and as a result, the grasping function and the closing function as described above can be surely exhibited.

As shown in FIG. 4C, the upper end section 28 of the disposal bag. 12 is returned to its original, vertically extended condition from the reversed, covering condition.

Then, by pulling the string 31, the open edge 27 of the disposal bag 12 is closed. As shown in FIG. 4E, the mouth periphery 23 of the disposal bag 12 is tied up from outside using the extended section of the string 31 and is closed.

(10) In this manner, the disposal bag 12 housing the object is removed from the excrement disposal device 8 (the double-faced adhesive tape is removed, if necessary). As shown in FIG. 4F, the disposal bag 12 is housed in a separate outer bag J, and any remaining air is expelled from the outer bag J.

The outer bag J is made of a non-porous material of which the odor-eliminating property is good. For example, a bag of aluminum foil is used.

As shown in FIG. 4G, the outer bag J in which the disposal bag 12 housing the object is housed, is sealed off using a seal adhesive and is scrapped.

Referring to the example as shown in FIG. 4, the disposal bag 12 housing the object is removed from the excrement disposal device 8. However, everything can be thrown away. Namely, the excrement disposal device 8 with the disposal bag 12 housing the object can be housed in the outer bag J, sealed off and discarded.

### EFFECTS OF THE INVENTION

(First Effect)

According to this excrement disposal device, it is possible to correctly and readily apply a mouth section to the subanal area for positioning.

In other words, in the excrement disposal device according to the present invention, by inserting the fingertips into the right and left spaces of the mouth section, it is possible to locate the anus with the fingertips through the disposal bag of the mouth periphery for confirmation and positioning before the fingertips are pushed against the anus. Namely, the fingertips can directly reach the anus through the mouth periphery of the disposal bag and, based on the mutual direct feel, i.e., the tactile impression, between the fingertips and the anus located by the fingertips, it is possible to readily realize the operation for correctly matching the center of the excrement disposal device, the center of the mouth section, and the center of the disposal bag of the mouth periphery with the center of the anus for positioning.

In this manner, the disadvantage as seen in the conventional excrement disposal device of this type whereby the location of the anus cannot be correctly grasped and specified, causing the excrement disposal device to be displaced from the anus, thereby producing a gap, can be surely prevented.

Thus, an accident whereby an object escapes from the mouth section and the disposal bag can be eliminated, and the object can be surely housed in the disposal bag. In this manner, the problems such as adhesion (adherence) of the object to the body, soil and odor can be surely eliminated and solved to greatly improve the cleanliness.

(Second Effect)

According to this excrement disposal device, the operation is stable and the mouth section is adapted to open and close in accordance with the opening and closing motion of the anus.

Namely, in the excrement disposal device according to the present invention, the mouth section formed by the opening of the base plates is controlled to make the size variable by the fingertips pushed against the anus area and the wing plates. With this, the mouth periphery of the disposal bag can be formed in a substantially circular-ring shape to perfectly match the shape of the anus. When the anus is opened or closed in excretion, the fingertips playing an important role in the opening and closing motion of the anus move to allow the mouth section to open or close according to the anus. Thus, the mouth periphery of the disposal bag can be stably and surely formed in the substantially circular-ring shape to match the shape of the opening and closing anus.

Further, such a stable operation can be guaranteed and accelerated by factors such as the overlapping of the base plates, the curvature of the base plates, tongue pieces of the base plates, tying up of the mouth periphery of the disposal bag, and positioning of the fingertips in the center.

Thus, the situation as seen in the conventional excrement disposal device of this type described above, in which the mouth section does not move according to the opening and closing motion of the anus, is not caused and as a result, production of a displacement or gap can be surely prevented.

Even from this aspect, an accident whereby the object escapes from the mouth section and the disposal bag can be eliminated, and the object can be surely housed in the disposal bag. Accordingly, the problems such as adherence of the object to the body or fingertips, soiling and fecal odor can also be eliminated and solved to greatly improve the cleanliness, and the smell can be cut off as well. It is to be noted that the anus area can be made clean to such a degree that wiping out with paper or washing with water is not necessary and outflow of the odor can be surely prevented.

As described above, the present invention has a great effect in that all the problems of the conventional excrement disposal device of this kind can be solved.

1. An excrement disposal device comprising a pair of base plates, a pair of wing plates, and a disposal bag, wherein the pair of base plates is laterally provided and front and rear ends of the base plates are pivotally attached to each other to make dimensions of an opening variable between the two base plates; the pair of wing plates is vertically provided to be mounted on the two base plates, top sections of the front and rear wing plates are opposedly situated, facing the opening from ahead and from behind, to control the shape of the opening in a longitudinal direction, thereby making dimensions of a mouth section variable; and the disposal bag is vertically inserted into the mouth section and a mouth periphery of the inserted disposal bag is provided for touching a subanal area from below for positioning and being opened and closed in accordance with the opening and closing motion of the anus.

2. The excrement disposal device according to claim 1, wherein the two base plates are formed in a substantially long plate shape in the vertical direction to be bent upwards; at least the front and rear ends of the two base plates overlap at the pivotal attachment thereof to each other, and each base plate is provided with a cutaway section in the center; and the opening is formed by at least right and left cutaway sections and the dimensions of the opening are made variable based on a displacement of right and left base plates in a lateral direction, using a pivot point of the front and rear ends as a fulcrum.

3. The excrement disposal device according to claim 2, wherein each base plate has a central area, adjacent to the cutaway section, bent upwards in advance in a substantially convex shape.

4. The excrement disposal device according to claim 2, wherein each base plate is provided with a cut-in tongue piece near the front and rear ends; and each tongue piece is adapted to sandwich a base end section of each wing plate therebetween to allow the wing plate to slide along the base plate.

5. The excrement disposal device according to claim 1, wherein the front and rear wing plates are slightly bendable upwards to move back and forth while slidingly contacting each base plate, and one end of the base end section is pivotally attached to one of the right and left base plates, while another end of the base end section is pivotally attached to another end of the base plates; and the dimensions of the mouth section are made variable by displacing both base plates in the lateral direction, using the pivot point of the front and rear ends of each base plate as a fulcrum, and by allowing each top section of both wing plates to longitudinally approach or recede in conjunction with the displacement of the base plates.

6. The excrement disposal device according to claim 1, wherein the disposal bag is detachably mounted on the excrement disposal device, an upper end section of which the edge is open is reversed to cover both base plates and wing plates, and a bottom section is provided to cause an object excreted from the anus to flow down through the mouth periphery for housing the object.

7. The excrement disposal device according to claim 6, wherein the disposal bag is made of a flexible resin material and a waist body is attached to the disposal bag from the mouth periphery toward the bottom section to lower the flexibility of the disposal bag in the area where the waist body is attached and to facilitate the downward flow of the object.

8. The excrement disposal device according to claim 1, wherein: right and left spaces of the mouth section are provided each to receive a fingertip, the disposal bag of the mouth periphery controls an enclosure whereby the inserted fingertips do not project too far upward, and the mouth section is controlled between the inserted right and left fingertips and the top sections of the front and rear wing plates to make the lateral dimensions and the vertical dimensions variable; and the mouth periphery of the disposal bag is formable in a substantially circular-ring shape to match the shape of the anus.

9. The excrement disposal device according to claim 8, wherein: a finger-inserting section is respectively attached to the underside of a cutaway section of the two base plates to cause the fingertips to be inserted therein from the bottom up and to cause the inserted fingertips to be pushed against the anus directly from below through the disposal bag of the mouth periphery, and the finger-inserting section laterally displaces both base plates by the movement of the inserted fingertips moving in accordance with the opening and closing motion of the anus, in the lateral direction, using the pivotal points of the front and rear ends of both base plates as a fulcrum, and make both the lateral and vertical dimensions of the mouth section variable, thereby causing the disposal bag of the mouth periphery to open and close.
